(19) United States
(12) Patent Application Publication
(10) Pub. No.: US 2004/0131631 A1
(43) Pub. Date: Jul. 8, 2004

(54) Polyphosphazene immunostimulants

(76) Inventors: Alexander Andrianov, Belmont, MA (US); Bryan Roberts, Cambridge, MA (US)

Correspondence Address: Raymond E. Stauffer, Esq., c/o Carella, Byrne, Bain, Gilfillan,, Cecchi, Stewart & Olstein, 6 Becker Farm Road, Roseland, NJ, 07068, US

(21) Appl. No.: 10/715,788

(22) Filed: Nov. 18, 2003

Related U.S. Application Data
(60) Provisional application No. 60/428,416, filed on Nov. 22, 2002.

(51) Int. Cl. C08G 63/48; C08G 63/91; A61K 39/00

(52) U.S. Cl. 424/184.1; 525/54.1

(57) ABSTRACT

Polyphosphazenes suitable for use as immunostimulants alone or in combination with an antigen are disclosed.

This invention relates to polyphosphazenes. The present invention further relates to polyphosphazenes suitable for use as an immunostimulant alone or in combination with an antigen, and to the use thereof.

In accordance with one aspect to the present invention, there is provided a polymer that comprises a polyphosphazene backbone and pendant or side groups wherein at least a portion of the pendant or side groups are capable of binding to receptors on immune cells.

In accordance with a further aspect of the present invention, there is provided a polymer comprising a polyphosphazene backbone having pendant or side groups wherein at least a portion of the pendant groups bind to receptors on immune cells that activates innate immunity and/or induces Th1 activated acquired immunity (cell mediated responses).

In a preferred embodiment of the invention, at least a portion of the pendant groups of the polyphosphazene backbone includes a saccharide that is capable of binding to a mannose-receptor on an immune cell, which may be a monosaccharide or polysaccharide.

The innate immunity system is stimulated through the activation of receptors, which are sometimes referred to as pattern recognition receptors (PRR).

In most cases, such receptors are activated as they bind pathogens through the recognition of pathogen associated molecular patterns (PAMP). The receptors that activate the innate system include the leucine rich proteins that form the so called Toll Receptors that bind and are activated by lipopolysaccharides (LPS), lipoteichoic acids (LTA), glycolipids, glucose and mannose containing polysaccharides, such as yeast mannans and zymosan, unmethylated CpG motifs such as those in bacterial DNA and double stranded RNA viruses. There are also receptors with extracellular C-type Lectin-like domain that recognize the terminal mannose and glucose of oligosaccharides of pathogen origin. Such receptors include the mannose receptor on macrophages and dendritic cells, DEC205 on dendritic cells and thymic epithelium, and the glucan binding DECTIN-1 receptor on the surface of macrophages and dendritic cells.

Thus, in accordance with an aspect of the present invention, there is provided a polymer that includes a polyphosphazene backbone having pendant or side groups wherein at least a portion of the side groups recognize or bind to a receptor for activating the innate system or induces Th1 activated acquired immunity. More particularly, and in a preferred aspect, the present invention provides such a polyphosphazene with pendant or side groups wherein at least a portion of such groups bind to a mannose receptor on immune cells (preferably human immune cells) and preferably a mannose receptor that activates the innate system.

In one aspect of the present invention, wherein the polyphosphazene contains pendant or side groups wherein at least a portion thereof binds to a mannose receptor on immune cells, such pendant group may be a saccharide which may be a monosaccharide or an oligosaccharide or a polysaccharide. In a preferred embodiment, such saccharide (in the form of a monosaccharide or terminal saccharide of an oligosaccharide) is one which contains mannose, fucose, N-actylglucosamine or glucose. Such monosaccharide or terminal saccharide of an oligosaccharide may be, for example, mannose, glucose, GlcNAc, fucose, galactose, GalNAc, and mannose, but it is to be understood that the present invention is not limited to such preferred materials.

The saccharide may be linked to the polyphosphazene backbone directly by use of one of the hydroxyl groups on the oligosaccharide or monosaccharide, or may be linked to the polymer backbone through a suitable linker or spacer group. Thus, for example, such a linker group may be an alkylene group, which preferably contains from 1 to 20 carbon atoms or may be an alkyleneoxy group that contains from 1 to 20 carbon atoms. Alternatively, saccharide can be linked to polyphosphazene through non-covalent bonds, such as ionic, hydrogen bonds, or hydrophobic associations. Thus, for example saccharide can be linked to a polyphosphazene polyelectrolyte as a counterion.

The polymer backbone may include the same saccharide pendant or side groups or may include two or more different saccharide side groups.

In accordance with another embodiment of the present invention, the polyphosphazene includes pendant or side groups, at least a portion of which bind to a toll receptor for the innate immunity system. As representative examples of ligands that bind to toll receptors, there may be mentioned viral double stranded RNA, LPS (lipopolysaccharide), LPA (lipoteichoic acid), bacterial flagella, and CpG.

The polyphosphazene may include pendant groups or side groups in addition to those of the type hereinabove described that bind to a receptor on human cells. Thus, for example, the polyphosphazene may include pendant groups that will function to increase binding of the receptor binding side groups. As representative examples of such groups, there may be mention strong acids groups, such as sulfonic acids or phosphonic acid, and other groups such as amino acid side groups (which may be in the form of a peptide) or lipid groups. The sulfonic acid or phosphonic acid groups are present on an organic moiety attached as a pendant group to the polymer backbone; for example oxyphenyl SO3-, oxyalkyl SO3-, etc.

In addition, in order to facilitate binding to the receptor, side groups may be introduced into the polyphosphazene that increase the flexibility of the polymer chain. As representative examples of such side groups, there may be mentioned alkoxy, (preferably an alkoxy group that contains from 1 to 4 carbon atoms), or aminoalkyl, (preferably one that contains from 1 to 4 carbon atoms), or an alkyl group, (preferably one that contains from 1 to 4 carbon atoms).

The polyphosphazene may also include side groups, such as a polyelectrolytic side group that produces binding of an antigen thereto, particularly in the case where the polyphosphazene immunostimulant is to be used in combination with an antigen. As representative groups which may be employed as such pendant or side groups are those that can be ionized i.e. a polyelectroyltic side group such as a carboxylatophenoxy side group.

The polyphosphazenes of the present invention may be employed as a water soluble polymer, or may be used in the form of a microsphere. Methodology for producing such polyphosphazenes in the form of a microsphere is described, for example, in U.S. Pat. No. 5,807,757. Thus, the immunostimulant of the invention with the hereinabove described pendant or side groups may be used in either a soluble form or a particulate form.

Thus, the polymers of the present invention include a polyphosphazene that include pendant groups all of which or some of which are capable of binding to receptors on immune cells that activate the innate immunity system, with such pendant groups preferably being those that bind to a mannose receptor on immune cells. The receptor binding pendant group may all be the same groups or may be two or more different groups (when the polymer backbone includes two or more different groups the polymer is a copolymer). The polymers preferably have a molecular weight of at least about 10,000 g/mol.

As hereinabove indicated, the polymer may also include pendant groups in addition to the pendant groups that bind to a receptor of the type hereinabove described.

The polymers of the present invention may be employed to stimulate the immune system and may be used alone or in combination with an antigen. When used in combination with an antigen the polymer potentiates the immune response to the antigen by binding to a receptor of the innate system. Thus, in accordance with an embodiment of the invention there is provide a pharmaceutical composition that includes a polyphosphazene of the type hereinabove described and an antigen for inducing an immune response. The composition contains an amount of polyphosphazene that is effective to activate the innate immune system and an amount of antigen effective to produce an immune response, in particular a Th1 immune response.

When used in combination with an antigen, in addition to pendant groups that bind to a receptor as described, the polymer backbone preferably includes pendant groups that are ionizable to produce a polyphosphazene polyelectrolyte. Such a polyelectrolyte complexes with the antigen.

The polymers of the present invention may be produced by initially producing poly (dichlorophosphazene). The pendant groups are then substituted onto the polymer backbone by reaction between the reactive chloro-groups on the backbone and the appropriate organic compound. For example, a saccharide may be reacted with the reactive chlorine atoms on the polymer backbone through a hydroxyl group of the saccharide by procedures known in the art. In such a reaction, there is selective protection of hydroxyl groups that are not to react with the chlorine atoms and activation of one of the hydroxyl groups for reaction. When using an oligosaccharide, the pendant groups include a terminal saccharide that binds to the receptor.

When using the polymer in combination with an antigen, the antigen may be any one of a wide variety of antigens against which an immune response is desired, and in particular an immunoprotective response. The immunogenic response may be humoral and/or cell mediated and in particular a cell mediated response.

The polymer alone or in combination with an antigen is used in an amount effective to provide the desired immune response. In general, the polymer alone or in combination with antigen is employed in a pharmaceutically effective carrier. The immunogenic composition can be administered as a vaccine by any method known to those skilled in the art that elicits an immune response; including parenterally, orally, or transmembrane or transmucosal administration. Preferably, the vaccine is administered parenterally (intravenously, intramuscularly, subcutaneously, Non-limiting examples of routes of delivery to mucosal surfaces are intranasal (or generally, the nasal associated lymphoid tissue), respiratory, vaginal and rectal.

The polymers of the invention when employed in the absence of antigen activate the immune system and may be employed to provide a non-specific immune response against pathogens; and in particular, may be used for protective purposes.

What is claimed is:

1. A polymer comprising a polyphosphazene backbone having pendant groups, at least a portion of the pendant groups of the polyphosphazene backbone bind to a receptor on human cells that activates innate immunity.

2. A polymer comprising a polyphosphazene backbone having pendant groups, at least a portion of the pendant groups of the polyphosphazene backbone binding to a receptor on human cells that activates an antigen specific Th1 immune response.

3. A composition containing the polymer of claim 2 and an antigen for the induction of antigen specific Th1 immune response.
